(19) United States
(12) Patent Application Publication
(10) Pub. No.: US 2022/0041694 A1
(43) Pub. Date: Feb. 10, 2022

(54) SARS-COV-2 ANTIBODIES FOR TREATMENT AND PREVENTION OF COVID-19

(71) Applicant: AstraZeneca UK Limited, Cambridge, MA (US)

(72) Inventors: Robert GASSER, Gaithersburg, MD (US); Mark ESSER, Gaithersburg, MD (US); Patrick MCTAMNEY, II, Gaithersburg, MD (US); Yueh-Ming LOO, Gaithersburg, MD (US); Reena M. VARKEY, Gaithersburg, MD (US); Qun DU, Gaithersburg, MD (US); James STEINHARDT, Gaithersburg, MD (US); Saravanan RAJAN, Gaithersburg, MD (US)

(21) Appl. No.: 17/397,203

(22) Filed: Aug. 9, 2021

Related U.S. Application Data
(60) Provisional application No. 63/063,862, filed on Aug. 10, 2020.
(60) Provisional application No. 63/112,104, filed on Nov. 10, 2020.

(51) Int. Cl. C07K 16/10; A61P 31/14; A61K 9/00

(52) U.S. Cl. CPC C07K 16/10; A61K 2039/507; A61K 9/0019; A61P 31/14

(57) ABSTRACT

The present disclosure provides antibodies and antigen-binding fragments thereof that specifically bind to the spike protein of SARS-CoV-2 and methods of using such antibodies and antigen-binding fragments thereof for the prevention and treatment of Coronavirus Disease 2019 (COVID-19) in a subject.

### 1. CROSS-REFERENCE TO RELATED APPLICATIONS

This application claims the priority benefit of U.S. Provisional Application No. 63/063,862, filed on Aug. 10, 2020, and U.S. Provisional Application No. 63/112,104, filed on Nov. 10, 2020, each of which is incorporated herein by reference in its entirety.

### 2. REFERENCE TO SEQUENCE LISTING SUBMITTED ELECTRONICALLY

The content of the electronically submitted sequence listing in ASCII text file (Name: 2943_1650003_Seqlisting_ST25; Size: 32,733 bytes; and Date of Creation: Jul. 23, 2021) filed with the application is incorporated herein by reference in its entirety.

### 3. FIELD

The present disclosure relates to relates generally to methods of using antibodies and antigen-binding fragments thereof for the prevention and treatment of Coronavirus Disease 2019 (COVID-19) in a subject.

### 4. BACKGROUND

Coronavirus 2019 (COVID 19), the disease caused by severe acute respiratory syndrome coronavirus 2 (SARS-CoV-2), has become a global pandemic. As of July 2020, over seventeen million cases and six hundred thousand deaths due to COVID-19 have been confirmed globally. The virus is capable of person-to-person spread through small droplets from the nose or mouth, which are expelled when an infected person coughs, sneezes, or speaks. The incubation period (time from exposure to onset of symptoms) ranges from 0 to 24 days, with a mean of 3-5 days, but it may be contagious during this period after recovery. Most people who contract SARS-CoV-2 show symptoms within 11.5 days of exposure, including fever, coughing, and breathing difficulties. The virus has a greater impact on patients of advanced age, with type 2 diabetes, cardiac disease, chronic obstructive pulmonary disease (COPD), and/or obesity. Most patient contracting the virus have mild symptoms, but in some patients, the infection in the lung is severe causing severe respiratory distress or even death.

There is currently no approved vaccine and no specific treatment that has garnered approval of the scientific and medical community, although several vaccine and antiviral approaches are being investigated. For example, because human monoclonal antibodies (mAbs) to the viral surface spike (S) glycoprotein mediate immunity to other coronaviruses including severe acute respiratory syndrome (SARS) and Middle East respiratory syndrome (MERS), it has been hypothesized that human mAbs targeting SARS-CoV-2 spike proteins may have promise for use in the prevention and treatment of SARS-CoV-2 infection. There is an urgent need for methods of preventing and treating COVID-19.

### 5. SUMMARY

Provided herein is a method of treating or preventing Coronavirus Disease 2019 (COVID-19) in a subject, the method comprising administering to a subject in need thereof about 300 mg to about 3000 mg of a first antibody or antigen-binding fragment thereof that binds to a spike protein of SARS-CoV-2 and about 300 mg to about 3000 mg of a second antibody or antigen-binding fragment thereof that binds to a spike protein of SARS-CoV-2, wherein the first antibody or antigen-binding fragment thereof comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and the second antibody or antigen-binding fragment thereof comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

In some aspects, the method comprises administering about 150 mg of the first antibody or antigen-binding fragment thereof and about 150 mg of the second antibody or antigen-binding fragment thereof. In some aspects, the method comprises administering about 300 mg of the first antibody or antigen-binding fragment thereof and about 300 mg of the second antibody or antigen-binding fragment thereof. In some aspects, the method comprises administering about 500 mg of the first antibody or antigen-binding fragment thereof and about 500 mg of the second antibody or antigen-binding fragment thereof. In some aspects, the method comprises administering about 1500 mg of the first antibody or antigen-binding fragment thereof and about 1500 mg of the second antibody or antigen-binding fragment thereof.

In some aspects, the first antibody or antigen-binding fragment thereof and the second antibody or antigen-binding fragment thereof are administered in separate pharmaceutical compositions.

In some aspects, the first antibody or antigen-binding fragment thereof and the second antibody or antigen-binding fragment thereof are administered sequentially. In some aspects, the first antibody or antigen-binding fragment and the second antibody or antigen-binding fragment are administered on the same day. In some aspects, the first antibody or antigen-binding fragment is administered before the second antibody or antigen-binding fragment. In some aspects, the second antibody or antigen-binding fragment is administered before the first antibody or antigen-binding fragment.

In some aspects, the first antibody or antigen-binding fragment thereof is administered parenterally. In some aspects, the second antibody or antigen-binding fragment thereof is administered parenterally. In some aspects, the first antibody or antigen-binding fragment thereof and the second antibody or antigen-binding fragment thereof are administered parenterally.

In some aspects, the first antibody or antigen-binding fragment thereof is administered intravenously. In some aspects, the second antibody or antigen-binding fragment thereof is administered intravenously. In some aspects, the first antibody or antigen-binding fragment thereof and the second antibody or antigen-binding fragment thereof are administered intravenously.

In some aspects the first antibody or antigen-binding fragment thereof is administered via intravenous infusion. In some aspects the second antibody or antigen-binding fragment thereof is administered via intravenous infusion. In some aspects the first antibody or antigen-binding fragment thereof and the second antibody or antigen-binding fragment thereof are administered via intravenous infusion.

In some aspects, the first antibody or antigen-binding fragment thereof is administered via intravenous infusion at a rate of about 20 mg/minute. In some aspects, the second antibody or antigen-binding fragment thereof is administered via intravenous infusion at a rate of about 20 mg/minute. In some aspects, the first antibody or antigen-binding fragment thereof and the second antibody or antigen-binding fragment thereof are administered via intravenous infusion at a rate of about 20 mg/minute.

In some aspects, the first antibody or antigen-binding fragment thereof and the second antibody or antigen-binding fragment thereof are administered simultaneously.

In some aspects, the first antibody or antigen-binding fragment thereof is administered intramuscularly. In some aspects, the second antibody or antigen-binding fragment thereof is administered intramuscularly. In some aspects, the first antibody or antigen-binding fragment thereof and the second antibody or antigen-binding fragment thereof are administered intramuscularly.

In some aspects, the first antibody or antigen-binding fragment thereof is are administered via direct deltoid intramuscular injection. In some aspects, the second antibody or antigen-binding fragment thereof is are administered via direct deltoid intramuscular injection. In some aspects, the first antibody or antigen-binding fragment thereof and the second antibody or antigen-binding fragment thereof are administered via direct deltoid intramuscular injection.

In some aspects, the administration prevents COVID-19.

In some aspects, the subject does not have COVID-19 at the time of the administration, and the administration prevents or decreases the severity of one or more symptoms of COVID-19.

In some aspects, the subject has an increased risk of COVID-19. In some aspects, the subject is a healthcare worker.

In some aspects, the subject has been exposed to SARS-CoV-2. In some aspects, the subject does not have a known exposure to SARS-CoV-2.

In some aspects, the subject has symptomatic COVID-19 at the time of the administration, and the administration decreases the severity of one or more symptoms of COVID-19 or prevents increasing severity of one or more symptoms of COVID-19.

In some aspects, the one or more symptoms is selected from the group consisting of fever, dry cough, dyspnea, sore throat, fatigue, or a combination thereof.

In some aspects, the administration treats the COVID-19.

In some aspects, the administration results in a serum concentration of the first antibody or antigen-binding fragment there and/or the second antibody or antigen-binding fragment thereof that is sufficient to neutralization SARS-CoV-2.

In some aspects, the administration results in the accumulation of the first antibody or antigen-binding fragment thereof and/or the second antibody or antigen-binding fragment thereof in the nasal fluid of the subject.

In some aspects, the subject is human.

In some aspects, the first antibody or antigen-binding fragment thereof comprises a variable heavy chain (VH) comprising the amino acid sequence of SEQ ID NO:7 and a variable light chain (VL) comprising the amino acid sequence of SEQ ID NO:8. In some aspects, the second antibody or antigen-binding fragment thereof comprises a variable heavy chain (VH) comprising the amino acid sequence of SEQ ID NO:15 and a variable light chain (VL) comprising the amino acid sequence of SEQ ID NO:16. In some aspects, (i) the first antibody or antigen-binding fragment thereof comprises a variable heavy chain (VH) comprising the amino acid sequence of SEQ ID NO:7 and a variable light chain (VL) comprising the amino acid sequence of SEQ ID NO:8 and (ii) second antibody or antigen-binding fragment thereof comprises a variable heavy chain (VH) comprising the amino acid sequence of SEQ ID NO:15 and a variable light chain (VL) comprising the amino acid sequence of SEQ ID NO:16.

In some aspects, the first antibody or antigen-binding fragment thereof comprises a heavy chain constant region. In some aspects, the second antibody or antigen-binding fragment thereof comprises a heavy chain constant region. In some aspects, the first antibody or antigen-binding fragment thereof and the second antibody or antigen-binding fragment thereof comprises a heavy chain constant region. In some aspects, the heavy chain constant region is a human IgG1 heavy chain constant region.

In some aspects, the first antibody or antigen-binding fragment thereof comprises a light chain constant region. In some aspects, the second antibody or antigen-binding fragment thereof comprises a light chain constant region. In some aspects, the first antibody or antigen-binding fragment thereof and the second antibody or antigen-binding fragment thereof comprises a light chain constant region. In some aspects, the light chain constant region is selected from the group consisting of human IgGκ and IgGλ light chain constant regions.

In some aspects, the first antibody or antigen-binding fragment thereof comprises a heavy chain constant region comprising a YTE mutation. In some aspects, the second antibody or antigen-binding fragment thereof comprises a heavy chain constant region comprising a YTE mutation. In some aspects, the first antibody or antigen-binding fragment and the second antibody or antigen-binding fragment thereof comprise a heavy chain constant region comprising a YTE mutation.

In some aspects, the first antibody or antigen-binding fragment thereof comprises a heavy chain constant region comprising a TM mutation. In some aspects, the second antibody or antigen-binding fragment thereof comprises a heavy chain constant region comprising a TM mutation. In some aspects, the first antibody or antigen-binding fragment and the second antibody or antigen-binding fragment thereof comprise a heavy chain constant region comprising a TM mutation.

In some aspects, the first antibody or antigen-binding fragment thereof comprises a heavy chain constant region comprising a YTE mutation and a TM mutation and the second antibody or antigen-binding fragment thereof comprises a heavy chain constant region comprising a YTE mutation and a TM mutation.

In some aspects, the first antibody or antigen-binding fragment thereof comprises a heavy chain comprising amino acids 1-460 of SEQ ID NO:24 and a light chain comprising the amino acid sequence of SEQ ID NO:25. In some aspects, the second antibody or antigen-binding fragment thereof comprises a heavy chain comprising amino acids 1-460 of SEQ ID NO:22 and a light chain comprising the amino acid sequence of SEQ ID NO:23. In some aspects, (i) the first antibody or antigen-binding fragment thereof comprises a heavy chain comprising amino acids 1-460 of SEQ ID NO:24 and a light chain comprising the amino acid sequence of SEQ ID NO:25 and (ii) the second antibody or antigen-binding fragment thereof comprises a heavy chain comprising amino acids 1-460 of SEQ ID NO:22 and a light chain comprising the amino acid sequence of SEQ ID NO:23.

In some aspects, the first antibody or antigen-binding fragment thereof comprises a heavy chain comprising the amino acid sequence of SEQ ID NO:24 and a light chain comprising the amino acid sequence of SEQ ID NO:25. In some aspects, the second antibody or antigen-binding fragment thereof comprises a heavy chain comprising the amino acid sequence of SEQ ID NO:22 and a light chain comprising the amino acid sequence of SEQ ID NO:23. In some aspects, (i) the first antibody or antigen-binding fragment thereof comprises a heavy chain comprising the amino acid sequence of SEQ ID NO:24 and a light chain comprising the amino acid sequence of SEQ ID NO:25 and (ii) the second antibody or antigen-binding fragment thereof comprises a heavy chain comprising the amino acid sequence of SEQ ID NO:22 and a light chain comprising the amino acid sequence of SEQ ID NO:23.

In some aspects, the first antibody or antigen-binding fragment thereof is fully human. In some aspects, the second antibody or antigen-binding fragment thereof is fully human. In some aspects, the first antibody or antigen-binding fragment and the second antibody or antigen-binding fragment thereof are fully human.

In some aspects, the first antibody or antigen-binding fragment thereof is humanized. In some aspects, the second antibody or antigen-binding fragment thereof is humanized. In some aspects, the first antibody or antigen-binding fragment and the second antibody or antigen-binding fragment thereof are humanized.

In some aspects, the first antibody or antigen-binding fragment thereof is a full length antibody. In some aspects, the second antibody or antigen-binding fragment thereof is a full length antibody. In some aspects, the first antibody or antigen-binding fragment and the second antibody or antigen-binding fragment thereof are full length antibodies.

In some aspects, the first antibody or antigen-binding fragment thereof is an antigen-binding fragment. In some aspects, the second antibody or antigen-binding fragment thereof is an antigen-binding fragment. In some aspects, the first antibody or antigen-binding fragment and the second antibody or antigen-binding fragment thereof are antigen-binding fragments.

In some aspects, the first antigen-binding fragment comprises a Fab, Fab′, F(ab′)₂, single chain Fv (scFv), disulfide linked Fv, V-NAR domain, IgNar, IgGΔCH2, minibody, F(ab′)₃, tetrabody, triabody, diabody, single-domain antibody, (scFv)₂, or scFv-Fc. In some aspects, the second antigen-binding fragment comprises a Fab, Fab′, F(ab′)₂, single chain Fv (scFv), disulfide linked Fv, V-NAR domain, IgNar, IgGΔCH2, minibody, F(ab′)₃, tetrabody, triabody, diabody, single-domain antibody, (scFv)₂, or scFv-Fc. In some aspects, the first antigen-binding fragment and the second antigen-binding fragments comprise a Fab, Fab′, F(ab′)₂, single chain Fv (scFv), disulfide linked Fv, V-NAR domain, IgNar, IgGΔCH2, minibody, F(ab′)₃, tetrabody, triabody, diabody, single-domain antibody, (scFv)₂, or scFv-Fc.

In some aspects, the first antibody or antigen-binding fragment thereof is isolated. In some aspects, the second antibody or antigen-binding fragment thereof is isolated. In some aspects, the first antibody or antigen-binding fragment and the second antibody or antigen-binding fragment thereof are isolated.

In some aspects, the first antibody or antigen-binding fragment thereof is monoclonal. In some aspects, the second antibody or antigen-binding fragment thereof is monoclonal. In some aspects, the first antibody or antigen-binding fragment and the second antibody or antigen-binding fragment thereof are monoclonal.

In some aspects, the first antibody or antigen-binding fragment thereof is recombinant. In some aspects, the second antibody or antigen-binding fragment thereof is recombinant. In some aspects, the first antibody or antigen-binding fragment and the second antibody or antigen-binding fragment thereof are recombinant.

Also provided herein is a method of preventing or treating Coronavirus Disease 2019 in a subject, the method comprising: intravenously administering to the subject about 150 mg of a first antibody that specifically binds to a spike protein of SARS-CoV-2 and comprises: a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and intravenously administering to the subject about 150 mg of a second antibody that specifically binds to the spike protein of SARS-CoV-2 and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

Also provided herein is a method of preventing or treating Coronavirus Disease 2019 in a subject, the method comprising: intravenously administering to the subject about 300 mg of a first antibody that specifically binds to a spike protein of SARS-CoV-2 and comprises: a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and intravenously administering to the subject about 300 mg of a second antibody that specifically binds to the spike protein of SARS-CoV-2 and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

Also provided herein is a method of preventing or treating Coronavirus Disease 2019 in a subject, the method comprising: intravenously administering to the subject about 500 mg of a first antibody that specifically binds to a spike protein of SARS-CoV-2 and comprises: VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and intravenously administering to the subject about 500 mg of a second antibody that specifically binds to the spike protein of SARS-CoV-2 and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

Also provided herein is a method of preventing or treating Coronavirus Disease 2019 in a subject, the method comprising: intravenously administering to the subject about 1500 mg of a first antibody that specifically binds to a spike protein of SARS-CoV-2 and comprises: VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and intravenously administering to the subject about 1500 mg of a second antibody that specifically binds to the spike protein of SARS-CoV-2 and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

Also provided herein is a method of preventing or treating Coronavirus Disease 2019 in a subject, the method comprising: intramuscularly administering to the subject about 150 mg of a first antibody that specifically binds to a spike protein of SARS-CoV-2 and comprises: a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and intramuscularly administering to the subject about 150 mg of a second antibody that specifically binds to the spike protein of SARS-CoV-2 and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

Also provided herein is a method of preventing or treating Coronavirus Disease 2019 in a subject, the method comprising: intramuscularly administering to the subject about 300 mg of a first antibody that specifically binds to a spike protein of SARS-CoV-2 and comprises: a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and intramuscularly administering to the subject about 300 mg of a second antibody that specifically binds to the spike protein of SARS-CoV-2 and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

Also provided herein is a method of preventing or treating Coronavirus Disease 2019 in a subject, the method comprising: intramuscularly administering to the subject about 500 mg of a first antibody that specifically binds to a spike protein of SARS-CoV-2 and comprises: VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and intramuscularly administering to the subject about 500 mg of a second antibody that specifically binds to the spike protein of SARS-CoV-2 and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

Also provided herein is a method of preventing or treating Coronavirus Disease 2019 in a subject, the method comprising: intramuscularly administering to the subject about 1500 mg of a first antibody that specifically binds to a spike protein of SARS-CoV-2 and comprises: VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and intramuscularly administering to the subject about 1500 mg of a second antibody that specifically binds to the spike protein of SARS-CoV-2 and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14. In some aspects, the first antibody comprises a variable heavy chain (VH) comprising the amino acid sequence of SEQ ID NO:7 and a variable light chain (VL) comprising the amino acid sequence of SEQ ID NO:8; and the second antibody comprises a variable heavy chain (VH) comprising the amino acid sequence of SEQ ID NO:15 and a variable light chain (VL) comprising the amino acid sequence of SEQ ID NO:16.

In some aspects, the first antibody and the second antibody are each human IgG1 antibodies.

In some aspects, the first antibody comprises a human IgG1 constant region comprising a YTE mutation and a TM mutation and the second antibody comprises a human IgG1 constant region comprising a YTE mutation and a TM mutation.

In some aspects, the first antibody comprises a heavy chain comprising amino acids 1-460 of SEQ ID NO:24 and a light chain comprising the amino acid sequence of SEQ ID NO:25 and the second antibody comprises a heavy chain comprising amino acids 1-460 of SEQ ID NO:22 and a light chain comprising the amino acid sequence of SEQ ID NO:23.

In some aspects, the first antibody comprises a heavy chain comprising the amino acid sequence of SEQ ID NO:24 and a light chain comprising the amino acid sequence of SEQ ID NO:25 and the second antibody comprises a heavy chain comprising the amino acid sequence of SEQ ID NO:22 and a light chain comprising the amino acid sequence of SEQ ID NO:23.

In some aspects, the first antibody and the second antibody are administered sequentially on the same day. In some aspects, the first antibody is administered before the second antibody. In some aspects, the second antibody is administered before the first antibody.

### 6. BRIEF DESCRIPTION OF THE FIGURES

FIG. 1 shows a study flow chart of the Phase I trial described herein. (See Example 3.)

FIG. 2 shows a graphical representation of the Phase I trial described herein. (See Example 3.)

### 7. DETAILED DESCRIPTION

Provided herein are methods of using combinations of antibodies (e.g., monoclonal antibodies) and antigen-binding fragments thereof that specifically bind to the spike protein of SARS-CoV-2, e.g., for the treatment and prevention of COVID-19.

### 7.1 Terminology

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)₂, and Fv fragments, linear antibodies, and single chain antibodies. An antigen-binding fragment of an antibody can be derived from any animal species, such as rodents (e.g., mouse, rat, or hamster) and humans or can be artificially produced.

The terms “anti-SARS2-CoV-2 antibody,” “SARS-CoV-2 antibody” and “antibody that binds to SARS-CoV-2” are used interchangeably herein to refer to an antibody that is capable of binding to SARS-CoV-2. The extent of binding of a SARS-CoV-2 antibody to an unrelated, non-SARS-CoV-2 spike protein can be less than about 10% of the binding of the antibody to SARS-CoV-2 as measured, e.g., using ForteBio or Biacore. In some aspects provided herein, a SARS-CoV-2 antibody is also capable of binding to SARS-1. In some aspects provided herein, a SARS-CoV-2 antibody does not bind to SARS-1.

The terms “anti-spike protein of SARS2-CoV-2 antibody,” “SARS-CoV-2 spike protein antibody” and “antibody that binds to the spike protein of SARS-CoV-2” are used interchangeably herein to refer to an antibody that is capable of binding to the spike protein of SARS-CoV-2 with sufficient affinity such that the antibody is useful as a diagnostic and/or therapeutic agent in targeting SARS-CoV-2. The extent of binding of a SARS-CoV-2 spike protein antibody to an unrelated, non-SARS-CoV-2 spike protein can be less than about 10% of the binding of the antibody to SARS-CoV-2 spike protein as measured, e.g., using ForteBio or Biacore. In some aspects provided herein, a SARS-CoV-2 spike protein antibody is also capable of binding to the spike protein of SARS-1. In some aspects provided herein, a SARS-CoV-2 spike protein antibody does not bind to the spike protein of SARS-1.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The term “complementarity determining region” or “CDR” as used herein refers to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | (Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H02 | H95-H102 | H95-H102 |

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In some aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails some complementarity between the antigen-binding domain and the epitope. Accordingly, in some aspects, an antibody that “specifically binds” to the spike protein of SARS-CoV-2 can also bind to the spike protein of one or more related viruses (e.g., SARS-1) and/or can also bind to variants of the spike protein of SARS-CoV-2, but the extent of binding to an un-related, non-SARS-CoV-2 spike protein is less than about 10% of the binding of the antibody to the spike protein of SARS-CoV-as measured, e.g., using ForteBio or Biacore.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

As used herein, the term “host cell” can be any type of cell, e.g., a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer,” “administering,” “administration,” and the like, as used herein, refer to methods that may be used to enable delivery of a drug, e.g., a combination of antibodies or antigen-binding fragments thereof that specifically bind to the spike protein of SARS-CoV-2 to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human.

The term “therapeutically effective amount” refers to an amount of a drug, e.g., a combination of antibodies or antigen-binding fragments thereof effective to treat a disease or disorder in a subject.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. Patients or subjects in need of treatment can include those diagnosed with coronavirus 2019 (COVID-19) and those who have been infected with severe acute respiratory syndrome coronavirus 2 (SARS-CoV-2).

As used herein, the term “COVID-19” refers to an infection with SARS-CoV-2. A subject with COVID-19 can be symptomatic or asymptomatic.

Alternatively, the pharmacologic and/or physiologic effect may be prophylactic, i.e., the effect completely or partially prevents a disease or symptom thereof. In this respect, the disclosed method comprises administering a “prophylactically effective amount” of a drug (e.g., a combination of antibodies or antigen-binding fragments thereof). A “prophylactically effective amount” refers to an amount effective, at dosages and for periods of time necessary, to achieve a desired prophylactic result (e.g., prevention of COVID-19 or SARS-CoV-2 infection).

As used herein, the terms “combination” and “administered in combination” refer to the administration of one antibody or antigen-binding fragment thereof described herein with another antibody or antigen-binding fragment thereof described herein. The antibodies or antigen-binding fragments thereof in the combination can be administered simultaneously or sequentially. The antibodies or antigen-binding fragments thereof in the combination can be administered in the same or in different compositions.

As provided herein, reference to a “first” antibody or antigen-binding fragment thereof and a “second” antibody or antigen-binding fragment in a combination do not refer to the order of administration. The “first antibody or antigen-binding fragment thereof,” can be administered either before or after the “second antibody or antigen-binding fragment thereof.”

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided.

Any compositions or methods provided herein can be combined with one or more of any of the other compositions and methods provided herein.

### 7.2 Methods of Treatment Using A Combination of Anti-SARS-CoV-2 Antibodies or Antigen-Binding Fragments Thereof

Provided herein are methods of preventing COVID-19 (i.e., a SARS-CoV-2 infection) in a subject. Further provided herein are methods of treating COVID-19 in a subject. In some instances, the methods comprise administering a first and second anti-SARS-CoV-2 antibody or antigen-binding fragment thereof described herein or a pharmaceutical composition thereof as described herein to a subject in need thereof.

In some aspects, provided herein is a method of preventing COVID-19 in a subject, the method comprising administering to the subject about 300 mg to about 1500 mg of a first antibody or antigen-binding fragment thereof and about 300 mg to about 1500 mg of a second antibody or antigen-binding fragment thereof. The first antibody or antigen-binding fragment thereof can specifically binds to a spike protein of SARS-CoV-2 and comprise a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6. The second antibody or antigen-binding fragment thereof can specifically binds to a spike protein of SARS-CoV-2 and comprise a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

In some aspects, provided herein is a method of treating COVID-19 in a subject, the method comprising administering to the subject about 300 mg to about 1500 mg of a first antibody or antigen-binding fragment thereof and about 300 mg to about 1500 mg of a second antibody or antigen-binding fragment thereof. The first antibody or antigen-binding fragment thereof can specifically binds to a spike protein of SARS-CoV-2 and comprise a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6. The second antibody or antigen-binding fragment thereof can specifically binds to a spike protein of SARS-CoV-2 and comprise a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

In some aspects, the methods provided herein comprise administering to the subject about 500 mg to about 1500 mg of a first antibody or antigen-binding fragment thereof and about 500 mg to about 1500 mg of a second antibody or antigen-binding fragment thereof, optionally wherein the first antibody is administered before the second antibody.

In some aspects, the methods provided herein comprise administering to the subject about 150 mg to about 500 mg of a first antibody or antigen-binding fragment thereof and about 150 mg to about 500 mg of a second antibody or antigen-binding fragment thereof, optionally wherein the first antibody is administered before the second antibody.

In some aspects, the first antibody or antigen-binding fragment thereof and the second antibody or antigen-binding fragment thereof each bind to distinct, non-overlapping epitopes on the receptor binding domain (RBD) of the spike protein of SARS-CoV-2.

In some aspects, the methods provided herein comprise administering about 150 mg of the first antibody or antigen-binding fragment thereof and about 150 mg of the second antibody or antigen-binding fragment thereof. In some aspects, the methods provided herein comprise administering about 300 mg of the first antibody or antigen-binding fragment thereof and about 300 mg of the second antibody or antigen-binding fragment thereof. In some aspects, the methods provided herein comprise administering about 500 mg of the first antibody or antigen-binding fragment thereof and about 500 mg of the second antibody or antigen-binding fragment thereof. In some aspects, the methods provided herein comprise administering about 1500 mg of the first antibody or antigen-binding fragment thereof and about 1500 mg of the second antibody or antigen-binding fragment thereof.

In some aspects, the first antibody or antigen-binding fragment thereof and the second antibody or antigen-binding fragment thereof are administered separately. In some aspects, the first antibody or antigen-binding fragment thereof and the second antibody or antigen-binding fragment thereof are administered sequentially, e.g., by intravenous administration. In some aspects, the second antibody or antigen-binding fragment thereof is administered after the first antibody or antigen-binding fragment thereof. In some aspects, the first antibody or antigen-binding fragment thereof is administered after the second antibody or antigen-binding fragment thereof. In some aspects, the first antibody or antigen-binding fragment thereof and the second antibody or antigen-binding fragment thereof are administered sequentially on the same day (e.g., wherein one antibody or antigen-binding fragment thereof is administered within five hours, within four hours, within three hours, within two hours, within one hour, or within 30 minutes after administration of the other antibody or antigen-binding fragment thereof is complete).

In some aspects, the first antibody or antigen-binding fragment thereof and the second antibody or antigen-binding fragment thereof are administered simultaneously, e.g., a direct intramuscular injection of both.

In some aspects, the first antibody or antigen-binding fragment thereof is administered intramuscularly. In some aspects, the second antibody or antigen-binding fragment thereof is administered intramuscularly. In some aspects, the first antibody or antigen-binding fragment thereof and the second antibody or antigen-binding fragment thereof are administered intramuscularly. In some aspects, the administration is a direct deltoid intramuscular injection.

In some aspects, the first antibody or antigen-binding fragment thereof is administered parenterally. In some aspects, the second antibody or antigen-binding fragment thereof is administered parenterally. In some aspects, the first antibody or antigen-binding fragment thereof and the second antibody or antigen-binding fragment thereof are administered parenterally.

In some aspects, the first antibody or antigen-binding fragment thereof is administered intravenously. In some aspects, the second antibody or antigen-binding fragment thereof is administered intravenously. In some aspects, the first antibody or antigen-binding fragment thereof and the second antibody or antigen-binding fragment thereof are administered intravenously.

In some aspects, the first antibody or antigen-binding fragment thereof is administered via intravenous infusion. In some aspects, the second antibody or antigen-binding fragment thereof is administered via intravenous infusion. In some aspects, the first antibody or antigen-binding fragment thereof and the second antibody or antigen-binding fragment thereof are administered via intravenous infusion.

In some aspects, the first antibody or antigen-binding fragment thereof and the second antibody or antigen-binding fragment thereof are each administered via intravenous infusion at a rate of about 20 mg/minute.

### 7.3 Patient Population and Outcomes

Provided herein are clinical methods for preventing and treating COVID-19 (i.e., SARS-CoV-2 infection) in subjects (e.g., human subjects) using any method disclosed herein, for example, administering a first antibody or antigen-binding fragment thereof and a second antibody or antigen-binding fragment thereof, wherein the administration prevents or treats a SARS-CoV-2 infection.

In some aspects, the subject has an increased risk of SARS-CoV-2 infection. In some aspects, the subject is a healthcare worker. In some aspects, the subject has been exposed to SARS-CoV-2. In some aspects, the subject has not been exposed to SARS-CoV-2. In some aspects, the subject has an increased risk of SARS-CoV-2 infection. In some aspects, the subject is human.

In some aspects, a subject treated according to the methods disclosed herein preferably experience outcomes generally related to the prevention and treatment of COVID-19 (i.e., SARS-CoV-2 infection).

In some aspects, the methods disclosed herein, and the administration of the antibodies and antigen-binding fragments disclosed herein results in the prevention of one or more symptoms of COVID-19. In some aspects, the methods disclosed herein, and the administration of the antibodies and antigen-binding fragments disclosed herein administration results in the treatment of one or more symptoms of COVID-19. In some aspects, the symptoms comprise fever, dry cough, dyspnea, sore throat and/or fatigue.

In some aspects, the methods disclosed herein, and the administration of the antibodies and antigen-binding fragments disclosed herein results in the treatment of COVID-19.

In some aspects, the methods disclosed herein, and the administration of the antibodies and antigen-binding fragments disclosed herein administration results in accumulation of the first and/or second antibody or antigen-binding fragment thereof in the nasal fluid of the subject.

In some aspects, the methods disclosed herein, and the administration of the antibodies and antigen-binding fragments disclosed herein administration results in serum levels of the first and/or second antibody or antigen-binding fragments thereof sufficient to neutralize SARS-CoV-2.

In some aspects, the subject does not have COVID-19 (i.e., SARS-CoV-2 infection). In some aspects, the subject has COVID-19 but does not have symptoms of COVID-19. In some aspects, the subject has COVID-19 and has symptoms of the infection.

### 7.4 Antibodies and Antigen-Binding Fragments Thereof

In a specific aspect, provided herein are antibodies (e.g., monoclonal antibodies, such as human antibodies) and antigen-binding fragments thereof that bind to the spike protein of SARS-CoV-2. The amino acid sequence of the spike protein of SARS-CoV-2 is provided in SEQ ID NO:20:

(SEQ ID NO: 20)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFSAV
YAWNRKRISNCVADYSVLYNSASFSTFKCYGVSPTKLNDLCFTNVYADSF
VIRGDEVRQAIPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLPTPTWRVYSTGSNVFQTRAGC
LIGAEHVNNSYECDIPIGAGICASYQTQTNSPRRARSVASQSIIAYTMSL
GAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGG
FNFSQILPDPSKSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPCLKGVKLHYT

Amino acids 1-12 of SEQ ID NO:20 are the signal peptide of the spike protein. Therefore, the mature version of the spike protein of SARS-CoV-2 contains amino acids 13-1273 of SEQ ID NO:20. Amino acids 13-1213 of SEQ ID NO:20 correspond to the extracellular domain; amino acids 1214-1234 correspond to the transmembrane domain; and amino acids 1235-1273 correspond to the cytoplasmic domain.

In some aspects, an antibody or antigen-binding fragment thereof described herein, i.e., a first antibody or antigen-binding fragment thereof and/or a second antibody or antigen-binding fragment thereof, binds to the spike protein of SARS-CoV-2 and specifically binds to the receptor binding domain (RBD) of the spike protein of SARS-CoV-2.

In some aspects, the first antibody or antigen-binding fragment thereof described herein and the second antibody or antigen-binding fragment thereof described herein each bind to distinct, non-overlapping epitopes on the RBD of the spike protein of SARS-CoV-2.

In some aspects, the first antibody or antigen-binding fragment thereof described herein is antibody clone 2196. In some aspects, the second antibody or antigen-binding fragment thereof is antibody clone 2130.

In some aspects, an antibody or antigen-binding fragment thereof described herein, that specifically binds to the spike protein of SARS-CoV-2 cross-reacts with SARS-CoV. In some aspects, an antibody or antigen-binding fragment thereof described herein, that specifically binds to the spike protein of SARS-CoV-2 does not cross-react with SARS-CoV.

In some aspects, an antibody or antigen-binding fragment thereof described herein binds to the spike protein of SARS-CoV-2 and comprises the six CDRs of an antibody listed in Table 1 (i.e., the three VH CDRs of the antibody and the three VL CDRs of the same antibody).

TABLE 1
Antibody Sequences

| | SEQ | | CDR1 | CDR2 | CDR3 |
|---|---|---|---|---|---|
| Clone | ID NO | Sequence (Description) | (SEQ ID NO) | (SEQ ID NO) | (SEQ ID NO) |
| 2196 | 7 | QMQLVQSGPEVKKPGTSVKVSCKASGFTFMSSAVQWVRQARGQRLEWIG | GFTFMSSA | IVIGSGNT | AAPYCSSISCN |
| | | WIVIGSGNTNYAQKFQERVTITRDMSTSTAYMELSSLRSEDTAVYYCAA | (SEQ ID | (SEQ ID | DGFDI |
| | | PYCSSISCNDGFDIWGQGTMVTVSS | NO: 1) | NO: 2) | (SEQ ID |
| | | (Heavy chain variable region) | | | NO: 3) |
| 2196 | 8 | EIVLTQSPGTLSLSPGERATLSCRASQSVSSSYLAWYQQKPGQAPRLLI | QSVSSSY | GAS | QHYGSSRGWT |
| | | YGASSRATGIPDRFSGSGSGTDFTLTISRLEPEDFAVYYCQHYGSSRGW | (SEQ ID | (SEQ ID | (SEQ ID |
| | | TFGQGTKVEIK (Light chain variable region) | NO: 4) | NO: 5) | NO: 6) |
| 2196 | 24 | QMQLVQSGPEVKKPGSTVKSVSCKASGFTFMSSAVQWVRQARGQRLEWI | | | |
| | | GWIVIGSGNTNYAQKFQERVTITRDMSTSTAYMELSSLRSEDTAVYYCA | | | |
| | | APYCSSISCNDGFDIWGQGTMVTVSSASTKGPSVFPLAPSSKSTSGGTA | | | |
| | | ALGCLVKDYFPEPVTVSWNSGALTSGVHTFPAVLQSSGLYSLSSVVTVP | | | |
| | | SSSLGTQTYICNVNHKPSNTKVDKRVEPKSCDKTHTCPPCPAPEFEGGP | | | |
| | | SVFLFPPKPKDTLYITREPEVTCVVVSVSHEDPEVKFNWYVDGVEVHNA | | | |
| | | KTKPREEQYNSTYRVVSVLTVLHQDWLNGKEYKCKVSNKALPASIEKTI | | | |
| | | SKAKGQPREPQVYTLPPSREEMTKNQVSLTCLVKGFYPSDIAVEWESNG | | | |
| | | QPENNYKTTPPVLDSDGSFFLYSKLTVDKSRWQQGNVFSCSVMHEALHN | | | |
| | | HYTQKSLSLSPGK (Full length chain*; YTE | | | |
| | | underlined; TM bold and underlined) | | | |
| 2196 | 25 | EIVLTQSPGTLSLSPGERATLSCRASQSVSSSYLAWYQQKPGQAPRLLI | | | |
| | | YGASSRATGIPDRFSGSGSGTDFTLTISRLEDPEDFAVYYCQHYGSSRG | | | |
| | | WTFGQGTKVEIKRTVAAPSVFIFPPSDEQLKSGTASVVCLLNNFYPREA | | | |
| | | KVQWKVDNALQSGNSQESVTEQDSKDSTYSLSSTLTLSKADYEKHKVYA | | | |
| | | CEVTHQGLSSPVTKSFNRGEC (Full length light chain) | | | |
| 2130 | 15 | EVQLVESGGGLVKPGGSLRLSCAASGFTFRDVWMSWVRQAPGKGLEWVG | GFTFRDVW | IKSKIDGGTT | TTAGSYYYDTV |
| | | RIKSKIDGGTTDYAAPVKGRFTISRDDSKNTLYLQMNSLKTEDTAVYYC | (SEQ ID | (SEQ ID | GPGLPEGKFDY |
| | | TTAGSYYYDTVGPGLPEGKFDYWGQGTLVTVSS | NO: 9) | NO: 10) | (SEQ ID |
| | | (Heavy chain variable region) | | | NO: 11) |
| 2130 | 16 | DIVMTQSPDSLAVSLGERATINCKSSQSVLYSSNNKNYLAWYQQKPGQP | QSVLYSSNNKN | WAS | QQYYSTLT |
| | | PKLLMYWASTRESGVPDRFSGSGSGAEFTLTISSLQAEDVAIYYCQQYY | Y | (SEQ ID | (SEQ ID |
| | | STLTFGGGTKVEIK (Light chain variable region) | (SEQ ID | NO: 13) | NO: 14) |
| | | | NO: 12) | | |
| 2130 | 22 | EVQLVESGGGLVKPGGSLRLSCAASGFTFRDVWMSWVRQAPGKGLEWVG | | | |
| | | RIKSKIDGGTTDYAAPVKGRFTISRDDSKNTLYLQMNSLKTEDTAVYYC | | | |
| | | TTAGSYYYDTVGPGLPEGKFDYWGQGTLVTVSSASTKGPSVFPLAPSSK | | | |
| | | STSGGTAALGCLVKDYFPEPVTVSSWNSGALTSGVHTFPAVLQSSGLYS | | | |
| | | LSSVVTVPSSSLGTQTYICNVNHKPSNTKVDKRVEPKSCDKTHTCPPCP | | | |
| | | APEFEGGPSVFLFPPKPKDTLYITREPEVTCVVVDVSHEDPEVKFNWYV | | | |
| | | DGVEVHNAKTKPREEQYNSTYRVVSVLTVLHQDWLNGKEYKCKVSNKAL | | | |
| | | PASIEKTISKAKGQPREPQVYTLPPSREEMTKNQVSLTCLVKGFYPSDI | | | |
| | | AVEWESNGQPENNYKTTPPVLDSDGSFFLYSKLTVDKSRWQQGNVFSCS | | | |
| | | VMHHEALHNHYTQKSLSLSPGK (Full length heavy chain*; | | | |
| | | YTE underlined; TM bold and underlined) | | | |
| 2130 | 23 | DIVMTQSPDSLAVSLGERATINCKSSQSVLYSSNNKNYLAWYQQKPGQP | | | |
| | | PKLLMYWASTRESGVPDRFSGSGSGAEFTLTISSLQAEDVAIYYCQQYY | | | |
| | | STLTFGGGTKVEIKRTVAAPSVFIFPPSDEQLKSGTASVVCLLNNFYPR | | | |
| | | EAKVQWKVDNALQSGNSQESVTEQDSKDSTYSLSSTLTLSKADYEKHKV | | | |
| | | YACEVTHQGLSSPVTKSFNRGEC (Full length light chain) | | | |

*The full length heavy chain sequences provided in Table 1 contain a terminal lysine which can be
post-translationally clipped, so that the predominant form of the heavy chain can be amino acids 1-460
of SEQ ID NO: 22 and amino acids 1-460 of SEQ ID NO: 24.

In some aspects, the first antibody or antigen-binding fragment thereof described herein and the second antibody or antigen-binding fragment thereof described herein each bind to the spike protein of SARS-CoV-2 and comprise the two VH and two VL of the antibodies listed in Table 1.

In some aspects, the antibodies or antigen-binding fragments thereof described herein may be described by its 3 VL CDRs and/or or its 3 VH CDRs.

In some aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, provided herein are antibodies and antigen-binding fragments thereof that specifically bind to the spike protein of SARS-CoV-2 and comprise the Chothia VH and VL CDRs of the antibodies listed in Table 1. In some aspects, antibodies or antigen-binding fragments thereof that specifically bind to the spike protein of SARS-CoV-2 comprise one or more CDRs, in which the Chothia and Kabat CDRs have the same amino acid sequence. In some aspects, provided herein are antibodies and antigen-binding fragments thereof that specifically bind to the spike protein of SARS-CoV-2 and comprise combinations of Kabat CDRs and Chothia CDRs.

In some aspects, the CDRs of an antibody or antigen-binding fragment thereof can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In some aspects, provided herein are antibodies and antigen-binding fragments thereof that specifically bind to the spike protein of SARS-CoV-2 and comprise the IMGT VH and VL CDRs of an antibody listed in Table 1, for example, as described in Lefranc M-P (1999) supra and Lefranc M-P et al., (1999) supra).

In some aspects, the CDRs of an antibody or antigen-binding fragment thereof can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, provided herein are antibodies or antigen-binding fragments thereof that specifically bind to the spike protein of SARS-CoV-2 and comprise VH and VL CDRs of an antibody listed in Table 1 as determined by the method in MacCallum R M et al.

In some aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects, provided herein are antibodies or antigen-binding fragments thereof that specifically bind to the spike protein of SARS-CoV-2 and comprise VH and VL CDRs of an antibody listed in Table 1 as determined by the AbM numbering scheme.

In some aspects, provided herein are antibodies that comprise a heavy chain and a light chain. Non-limiting examples of human constant region sequences have been described in the art, e.g., see U.S. Pat. No. 5,693,780 and Kabat E A et al., (1991) supra.

With respect to the heavy chain, in some aspects, the heavy chain of an antibody described herein can be an alpha (α), delta (δ), epsilon (ε), gamma (γ) or mu (μ) heavy chain. In some aspects, the heavy chain of an antibody described can comprise a human alpha (α), delta (δ), epsilon (ε), gamma (γ) or mu (μ) heavy chain. In some aspects, an antibody described herein, which immunospecifically binds to the spike protein of SARS-CoV-2, comprises a heavy chain wherein the amino acid sequence of the VH domain comprises an amino acid sequence set forth in Table 1 and wherein the constant region of the heavy chain comprises the amino acid sequence of a human gamma (γ) heavy chain constant region (e.g., a human IgG1 heavy chain constant region). In some aspects, an antibody described herein, which specifically binds to the spike protein of SARS-CoV-2, comprises a heavy chain wherein the amino acid sequence of the VH domain comprises a sequence set forth in Table 1, and wherein the constant region of the heavy chain comprises the amino acid of a human heavy chain described herein or known in the art.

In some aspects, the light chain of an antibody or antigen-binding fragment thereof described herein is a human kappa light chain or a human lambda light chain. In some aspects, an antibody described herein, which immunospecifically binds to the spike protein of SARS-CoV-2 comprises a light chain wherein the amino acid sequence of the VL domain comprises a sequence set forth in Table 1 and wherein the constant region of the light chain comprises the amino acid sequence of a human kappa or lambda light chain constant region.

In some aspects, the antibodies or antigen-binding fragments thereof described herein, which immunospecifically bind to the spike protein of SARS-CoV-2 comprise a light chain wherein the amino acid sequence of the VL domain comprises a sequence set forth in Table 1, and wherein the constant region of the light chain comprises the amino acid sequence of a human kappa light chain constant region.

In some aspects, the light chain of an antibody described herein is a lambda light chain. In some aspects, an antibody described herein, which immunospecifically binds to the spike protein of SARS-CoV-2 comprises a light chain wherein the amino acid sequence of the VL domain comprises a sequence set forth in Table 1 and wherein the constant region of the light chain comprises the amino acid sequence of a human lambda light chain constant region.

In some aspects, the antibodies or antigen-binding fragments thereof described herein, which immunospecifically bind to the spike protein of SARS-CoV-2 comprise a VH domain and a VL domain comprising any amino acid sequence described herein, and wherein the constant regions comprise the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, an antibody described herein, which immunospecifically binds to the spike protein of SARS-CoV-2 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

Fc region engineering is used in the art, e.g., to extend the half-life of therapeutic antibodies and antigen-binding fragments thereof and protect from degradation in vivo. In some aspects, the Fc region of an IgG antibody or antigen-binding fragment can be modified in order to increase the affinity of the IgG molecule for the Fc Receptor-neonate (FcRn), which mediates IgG catabolism and protects IgG molecules from degradation. Suitable Fc region amino acid substitutions or modifications are known in the art and include, for example, the triple substitution M252Y/S254T/T256E (referred to as “YTE”) (see, e.g., U.S. Pat. No. 7,658,921; U.S. Patent Application Publication 2014/0302058; and Yu et al., Antimicrob. Agents Chemother., 61(1): e01020-16 (2017)). In some aspects, an antibody or antigen-binding binding fragment (e.g., monoclonal antibody or fragment) that binds to the spike protein of SARS-CoV-2 comprises an Fc region comprising the YTE mutation.

The triple mutation (TM) L234F/L235E/P331S (according to European Union numbering convention; Sazinsky et al. Proc Natl Acad Sci USA, 105:20167-20172 (2008)) in the heavy chain constant region can significantly reduce IgG effector function. In some aspects, an IgG1 sequence comprising the triple mutation comprises the of SEQ ID NO:21.

(SEQ ID NO: 21)
EPKSSDKTHTCPPCPAPEFEGGSPVFLFPPKPKDTLMISRTPECTCVVVD
VSHEDPEVKFNWYVDGVEVHNAKTKPREEQYNSTYRVVSVLTVLHQDWLN
GKEYKCKVSNKALPASIEKTISKAKGQPREPQVYTLPPSRDELTKNQVLS
TCLVKGFYPSDIAVEWESNGQPENNYKTTPPVLDSDGSFFLYSKLTVDKS
RWQQGNVFSCSVMHEALHNHYTQKSLSLSPGK

In some aspects, one, two, or more mutations (e.g., amino acid substitutions) are introduced into the Fc region of an antibody or antigen-binding fragment thereof described herein (e.g., into the CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody or antigen-binding fragment thereof, such as serum half-life, complement fixation, Fc receptor binding, and/or antigen-dependent cellular cytotoxicity.

In some aspects, one, two, or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain may be altered to, e.g., facilitate assembly of the light and heavy chains, or to alter (e.g., increase or decrease) the stability of the antibody or antigen-binding fragment thereof.

In some aspects, one, two, or more mutations (e.g., amino acid substitutions) are introduced into the Fc region of an antibody or antigen-binding fragment thereof described herein (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody or antigen-binding fragment thereof for an Fc receptor (e.g., an activated Fc receptor) on the surface of an effector cell. Mutations in the Fc region that decrease or increase affinity for an Fc receptor and techniques for introducing such mutations into the Fc receptor or fragment thereof are known to one of skill in the art. Examples of mutations in the Fc receptor that can be made to alter the affinity of the antibody or antigen-binding fragment thereof for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some aspects, one, two, or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody or antigen-binding fragment thereof in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody or antigen-binding fragment thereof in vivo. In some aspects, one, two or more amino acid mutations (i.e., substitutions, insertions, or deletions) are introduced into an IgG constant domain, or FcRn-binding fragment thereof (preferably an Fc or hinge-Fc domain fragment) to decrease the half-life of the antibody or antigen-binding fragment thereof in vivo. In some aspects, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody or antigen-binding fragment thereof in vivo. In some aspects, the antibodies or antigen-binding fragments thereof may have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some aspects, the constant region of the IgG1 comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall' Acqua WF et al., (2006) J Biol Chem 281: 23514-24). In some aspects, an antibody or antigen-binding fragment thereof comprises an IgG constant domain comprising one, two, three or more amino acid substitutions of amino acid residues at positions 251-257, 285-290, 308-314, 385-389, and 428-436, numbered according to the EU index as in Kabat.

In some aspects, one, two, or more amino acid substitutions are introduced into an IgG constant domain Fc region to alter the effector function(s) of the antibody or antigen-binding fragment thereof. For example, one or more amino acids selected from amino acid residues 234, 235, 236, 237, 297, 318, 320 and 322, numbered according to the EU index as in Kabat, can be replaced with a different amino acid residue such that the antibody or antigen-binding fragment thereof has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260. In some aspects, the deletion or inactivation (through point mutations or other means) of a constant region domain may reduce Fc receptor binding of the circulating antibody or antigen-binding fragment thereof thereby increasing tumor localization. See, e.g., U.S. Pat. Nos. 5,585,097 and 8,591,886 for a description of mutations that delete or inactivate the constant domain and thereby increase tumor localization. In some aspects, one or more amino acid substitutions can be introduced into the Fc region to remove potential glycosylation sites on Fc region, which may reduce Fc receptor binding (see, e.g., Shields R L et al., (2001) J Biol Chem 276: 6591-604).

In some aspects, one or more amino acids selected from amino acid residues 322, 329, and 331 in the constant region, numbered according to the EU index as in Kabat, can be replaced with a different amino acid residue such that the antibody or antigen-binding fragment thereof has altered C1q binding and/or reduced or abolished complement dependent cytotoxicity (CDC). This approach is described in further detail in U.S. Pat. No. 6,194,551 (Idusogie et al). In some aspects, one or more amino acid residues within amino acid positions 231 to 238 in the N-terminal region of the CH2 domain are altered to thereby alter the ability of the antibody to fix complement. This approach is described further in International Publication No. WO 94/29351. In some aspects, the Fc region is modified to increase the ability of the antibody or antigen-binding fragment thereof to mediate antibody dependent cellular cytotoxicity (ADCC) and/or to increase the affinity of the antibody or antigen-binding fragment thereof for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU index as in Kabat. This approach is described further in International Publication No. WO 00/42072.

In some aspects, the antibodies or antigen-binding fragments thereof described herein comprise the constant domain of an IgG1 with a mutation (e.g., substitution) at position 267, 328, or a combination thereof, numbered according to the EU index as in Kabat. In some aspects, an antibody or antigen-binding fragment thereof described herein comprises the constant domain of an IgG1 with a mutation (e.g., substitution) selected from the group consisting of S267E, L328F, and a combination thereof. In some aspects, an antibody or antigen-binding fragment thereof described herein comprises the constant domain of an IgG1 with a S267E/L328F mutation (e.g., substitution). In some aspects, an antibody or antigen-binding fragment thereof described herein comprising the constant domain of an IgG1 with a S267E/L328F mutation (e.g., substitution) has an increased binding affinity for FcγRIIA, FcγRIIB, or FcγRIIA and FcγRIIB.

Engineered glycoforms may be useful for a variety of purposes, including but not limited to enhancing or reducing effector function. Methods for generating engineered glycoforms in an antibody or antigen-binding fragment thereof described herein include but are not limited to those disclosed, e.g., in Umaña P et al., (1999) Nat Biotechnol 17: 176-180; Davies J et al., (2001) Biotechnol Bioeng 74: 288-294; Shields R L et al., (2002) J Biol Chem 277: 26733-26740; Shinkawa T et al., (2003) J Biol Chem 278: 3466-3473; Niwa Ret al., (2004) Clin Cancer Res 1: 6248-6255; Presta L G et al., (2002) Biochem Soc Trans 30: 487-490; Kanda Y et al., (2007) Glycobiology 17: 104-118; U.S. Pat. Nos. 6,602,684; 6,946,292; and 7,214,775; U.S. Patent Publication Nos. US 2007/0248600; 2007/0178551; 2008/0060092; and 2006/0253928; International Publication Nos. WO 00/61739; WO 01/292246; WO 02/311140; and WO 02/30954; Potillegent™ technology (Biowa, Inc. Princeton, N.J.); and GlycoMAb® glycosylation engineering technology (Glycart biotechnology AG, Zurich, Switzerland). See also, e.g., Ferrara C et al., (2006) Biotechnol Bioeng 93: 851-861; International Publication Nos. WO 07/039818; WO 12/130831; WO 99/054342; WO 03/011878; and WO 04/065540.

In some aspects, any of the constant region mutations or modifications described herein can be introduced into one or both heavy chain constant regions of an antibody or antigen-binding fragment thereof described herein having two heavy chain constant regions.

In some aspects, the first antibody or antigen-binding fragment thereof and the second antibody or antigen-binding fragment thereof each inhibit binding of SARS-CoV-2 to angiotensin converting enzyme 2 (ACE2).

In some aspects, the first antibody or antigen-binding fragment thereof and the second antibody or antigen-binding fragment thereof each neutralize SARS-CoV-2.

In some aspects, the first and second antigen-binding fragments disclosed herein comprise a Fab, Fab′, F(ab′)₂, single chain Fv (scFv), disulfide linked Fv, V-NAR domain, IgNar, IgGΔCH2, minibody, F(ab′)₃, tetrabody, triabody, diabody, single-domain antibody, (scFv)2, or scFv-Fc.

In some aspects, an antigen-binding fragment as described herein that specifically binds to the spike protein of SARS-CoV-2, is selected from the group consisting of a Fab, Fab′, F(ab′)₂, and scFv, wherein the Fab, Fab′, F(ab′)₂, or scFv comprises a heavy chain variable region sequence and a light chain variable region sequence of an antibody or antigen-binding fragment thereof described herein that specifically binds to the spike protein of SARS-CoV-2 or to SARS-CoV-2. A Fab, Fab′, F(ab′)₂, or scFv can be produced by any technique known to those of skill in the art. In some aspects, the Fab, Fab′, F(ab′)₂, or scFv further comprises a moiety that extends the half-life of the antibody in vivo. The moiety is also termed a “half-life extending moiety.” Any moiety known to those of skill in the art for extending the half-life of a Fab, Fab′, F(ab′)₂, or scFv in vivo can be used. For example, the half-life extending moiety can include a Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In some aspects, the Fab, Fab′, F(ab′)₂, or scFv can be modified by the addition of one or more C-terminal amino acids for attachment of the half-life extending moiety. In some aspects the half-life extending moiety is polyethylene glycol or human serum albumin. In some aspects, the Fab, Fab′, F(ab′)₂, or scFv is fused to a Fc region.

An antibody or antigen-binding fragment thereof that binds to the spike protein of SARS-CoV-2 can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labeled antibodies or antigen-binding fragments thereof can be used to detect the spike protein of SARS-CoV-2 or to SARS-CoV-2.

### 7.5 Pharmaceutical Compositions

Provided herein are methods of administering compositions comprising an anti-SARS-CoV-2 antibody or antigen-binding fragment thereof having the desired degree of purity in a physiologically acceptable carrier, excipient, or stabilizer (Remington's Pharmaceutical Sciences (1990) Mack Publishing Co., Easton, Pa.). Acceptable carriers, excipients, or stabilizers are nontoxic to recipients at the dosages and concentrations employed. (See, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). The compositions to be used for in vivo administration can be sterile. This is readily accomplished by filtration through, e.g., sterile filtration membranes.

In some aspects of the present disclosure, methods of administering pharmaceutical compositions are provided, wherein a first pharmaceutical composition comprises a first antibody or antigen-binding fragment thereof that specifically binds to a spike protein of SARS-CoV-2, and comprises: a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and a second pharmaceutical composition comprises a second antibody or antigen-binding fragment thereof that specifically binds the spike protein of SARS-CoV-2, and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

In some aspects, provided herein is a kit comprising (i) a first pharmaceutical composition comprising a first antibody or antigen-binding fragment thereof that specifically binds to a spike protein of SARS-CoV-2, and comprises: a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6 and (ii) a second pharmaceutical composition comprising a second antibody or antigen-binding fragment thereof that specifically binds to the spike protein of SARS-CoV-2, and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

In some aspects of the present disclosure, a pharmaceutical composition comprises (i) a first antibody or antigen-binding fragment thereof that specifically binds to a spike protein of SARS-CoV-2, and comprises: a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and (ii) a second pharmaceutical composition comprises a second antibody or antigen-binding fragment thereof that specifically binds to the spike protein of SARS-CoV-2, and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14. The pharmaceutical composition can further comprise a pharmaceutically acceptable excipient. In some aspects, the pharmaceutically acceptable excipient is not water.

In some aspects of the pharmaceutical compositions provided herein, the first antibody or antigen-binding fragment thereof comprises a VH comprising the amino acid sequence of SEQ ID NO:7 and/or a VL comprising the amino acid sequence of SEQ ID NO:8. In some aspects of the pharmaceutical compositions provided herein, the second antibody or antigen-binding fragment thereof comprises a VH comprising the amino acid sequence of SEQ ID NO:15 and/or a VL comprising the amino acid sequence of SEQ ID NO:16. In some aspects of the pharmaceutical compositions provided herein, the first antibody or antigen-binding fragment thereof comprises a VH comprising the amino acid sequence of SEQ ID NO:7 and/or a VL comprising the amino acid sequence of SEQ ID NO:8; and the second antibody or antigen-binding fragment thereof comprises a VH comprising the amino acid sequence of SEQ ID NO:15 and/or a VL comprising the amino acid sequence of SEQ ID NO:16.

In some aspects of the pharmaceutical compositions provided herein, the first antibody or antigen-binding fragment thereof is an IgG1 antibody, optionally wherein the IgG1 antibody comprises a YTE and/or TM mutation. In some aspects of the pharmaceutical compositions provided herein, the second antibody or antigen-binding fragment thereof is an IgG1 antibody, optionally wherein the IgG1 antibody comprises a YTE and/or TM mutation. In some aspects of the pharmaceutical compositions provided herein, the first antibody or antigen-binding fragment thereof is an IgG1 antibody, wherein the IgG1 antibody comprising a YTE and a TM mutation; and the second antibody or antigen-binding fragment thereof is an IgG1 antibody comprising a YTE and/or TM mutation

The following examples are offered by way of illustration and not by way of limitation.

### 8. EXAMPLES

### Example 1: Selection of Two Anti-SARS-CoV-2 Antibodies

The 2196+2130 antibodies were selected for use in a combination therapy (referred to as “2196+2130” herein). The 2196 and the 2130 antibodies that bind to distinct, non-overlapping sites on the receptor binding domain of the SARS-CoV-2 spike protein. Binding to either of these sites blocks the virus's ability to bind to its human cellular receptor, ACE2. By blocking virus entry into human cells, 2196+2130 can prevent or treat illness due to SARS-CoV-2 infection, COVID-19.

### Example 2: Selection of Dose of Two Anti-SARS-CoV-2 Antibodies

To support the selection of the study doses, a viral dynamic model was developed, which allows understanding of the pharmacodynamic effects of 2196+2130 on the growth of a SARS CoV-2 infection and the resulting immune response. For prophylaxis, the viral dynamic model indicates that virus entry inhibition greater than approximately 80% is sufficient to prevent infection. Assuming a partition ratio of 1% for lung endothelial lining fluid (ELF) to serum and an IC₅₀ of 26 ng/mL, a minimum effective dose of 245 mg intramuscular (IM) is required for prophylaxis to cover 5 months. If the partition ratio is lower at 0.1%, a conservative assumption, then a minimum effective dose of 2450 mg IM may be needed for prophylaxis. With 76% IM bioavailability assumed, IV doses would be proportionally lower. For subjects with active infection, the model indicates that virus entry inhibition greater than approximately 92% is sufficient to rapidly suppress the viral load to less than 1 copies per swab, which is a numerical cut-off criteria that is regarded as clinically meaningful effect that will accompany complete natural infection clearance. With an entry inhibition IC₅₀ of 26 ng/mL and a lung ELF-to-serum partition range of 1% to 0.1%, the minimum effective dose then ranges from 300 mg IM to 3000 mg IM. This aligns with the dose required to achieve the target reduction of 92% in virus entry inhibition. With 76% IM bioavailability assumed, IV doses would be proportionally lower.

### Example 3: Administration of Two Anti-SARS-CoV-2 Antibodies to Human Subjects

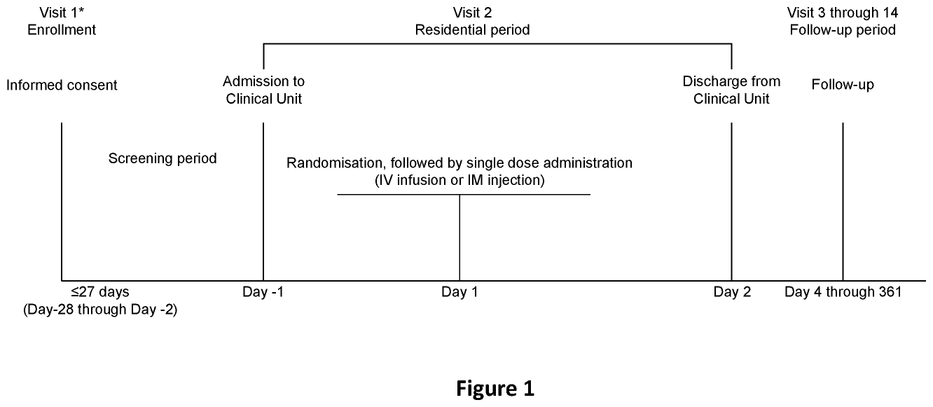

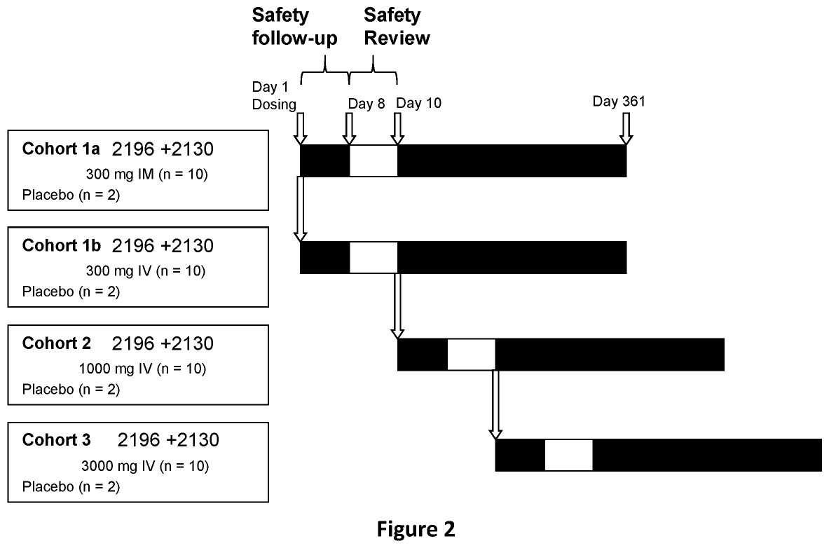

A Phase I, first time in human (FTIH), randomized, double-blind, placebo-controlled, dose escalation study is conducted to demonstrate the safety and tolerability of the sequential administration of two anti-SARS-CoV-2 antibodies (2196+2130) in healthy adult subjects 18 to 55 years of age. Approximately 48 subjects are enrolled and randomized 10:2 to either 2196+2130 or placebo administered via intravenous infusion or intramuscularly, across 4 fixed dose cohorts. The study flow chart is shown in FIG. 1, and a graphical representation is provided in FIG. 2. The study is approximately 389 days in duration for each subject, consisting of a Screening Period of up to 27 days (Day -28 through Day -2), a Treatment Period of 1 day (24 hours; Day 1) and a 360-day safety follow-up period (Day 361 post-dose).

During the screening period, each subject's medical history and demographics are obtained and a full physical examination is performed. A SARS-CoV-2 serology test is performed using quantitative real-time polymerase chain reaction (qRT-PCR), and eligibility criteria (the inclusion and exclusion criteria discussed below) are verified.

### Subjects

For inclusion in the study, subjects fulfill all of the following inclusion criteria and do not meet any of the exclusion criteria.

### Inclusion Criteria

1. Aged 18 through 55 years (both inclusive) at the time of screening.
2. Written informed consent.
3. Negative SARS-CoV-2 qRT-PCR and serology tests at screening.
4. Weight ≥50 kg and ≤110 kg at screening, including a BMI of ≥18.0 to ≤30.0 kg/m².
5. Healthy by medical history, physical examination, and baseline safety laboratory studies.
6. Electrocardiogram without clinically significant abnormalities at screening.
7. Able to complete the Follow-up Period through Day 361.

### Exclusion Criteria

1. Known hypersensitivity to any component of 2196+2130.
2. History of allergic disease or reactions likely to be exacerbated by any component of 2196+2130.
3. Previous hypersensitivity, infusion-related reaction or severe adverse reaction following administration of a monoclonal antibody.
4. Acute (time-limited) illness, including fever above 37.5° C. (99.5° F.), on day prior to or day of planned dosing; subjects excluded for transient acute illness may be dosed if illness resolves within the 27-day Screening Period or may be rescreened once.
5. Any drug therapy within 7 days prior to Day 1 (except contraceptives or a single use of acetaminophen, aspirin, antihistamine, or combination over-the-counter (OTC) product that contains acetaminophen with an antihistamine, or OTC nonsteroidal anti-inflammatory agent at a dose equal to or lower than that recommended on the package). Vitamins and other nutritional supplements that are not newly introduced, i.e., have been taken for at least 30 days prior to enrolment, are not exclusionary.
6. Blood drawn in excess of a total of 450 mL (1 unit) for any reason within 2 months prior to screening.
7. Receipt of immunoglobulin or blood products within 6 months prior to screening.
8. SARS-CoV-2 or COVID-19:
   a. Subjects with any confirmed current or previous COVID-19 infection before randomization.
   b. Subject has clinical signs and symptoms consistent with COVID-19, e.g., fever, dry cough, dyspnoea, sore throat, fatigue or confirmed infection by appropriate laboratory test within the last 4 weeks prior to screening or on admission.
   c. Any prior receipt of investigational or licensed vaccine indicated for the prevention of SARS-CoV-2 or COVID-19 or expected receipt during the period of study follow-up.
9. Receipt of any investigational product in the preceding 90 days or expected receipt of investigational product during the period of study follow-up, or concurrent participation in another interventional study.
10. Previous receipt of a monoclonal antibody within 6 months, or five antibody half-lives (whichever is longer), prior to study start.
11. Immunodeficiency due to illness, including HIV infection, or due to drugs, including any course of glucocorticoid therapy exceeding 2 weeks of prednisone or equivalent at a dose of 20 mg daily or every other day within 6 months prior to screening. HIV testing must be negative at screening.
12. Either history of active infection with hepatitis B or C or positive test for hepatitis C or for hepatitis B surface antigen at screening.
13. History of infection with Severe acute respiratory syndrome (SARS) or Middle East Respiratory Syndrome (MERS).
14. Aspartate aminotransferase, alanine aminotransferase (ALT), or serum creatinine above the upper limit of normal (ULN); bilirubin and alkaline phosphatase (ALP) >1.5×ULN.
15. Hemoglobin or platelet count below the lower limit of normal (LLN) at screening. White blood cell or neutrophil count outside normal references ranges.
16. History of malignancy.

### Restrictions During the Study

The following restrictions apply for the specified times during the study period:
1. Subjects rest comfortably during the IV infusion or lie supine for an hour after intramuscular administration. Subjects do not engage in any strenuous activity from 72 hours prior to check-in until discharge and 72 hours before every outpatient visit and their final follow-up visit.
2. Prior to the Treatment Period and outpatient visits, subjects abstain from alcohol for 72 hours prior to check-in until after their last pharmacokinetics (PK) sampling visit. Subjects also abstain from alcohol for 72 hours before their final follow-up visit.
3. Prior to the Treatment Period and outpatient visits, subjects abstain from caffeine-containing foods and beverages for 24 hours prior to check-in until discharge from the Clinical Unit.
4. During admission period, subjects receive a standard diet, which excludes all alcohol and caffeinated products.
5. Prior to admission, subjects avoid food containing poppy seeds for at least 72 hours.
6. Subjects are required to abstain from blood or plasma donation until 3 months after the final medical examination at the study follow-up.

### Sentinel Dosing

Dosing for all cohorts is initiated with 2 subjects in a sentinel cohort. Sentinel dosing is where one person in a first cohort of participants receives a single dose of a product in advance of the full study cohort.

One subject is randomized to receive a placebo and one subject is randomized to receive the 2196+2130 product. The safety data from the sentinel subjects up to 24 hours post-dose is reviewed before the remaining subjects in the cohort are dosed. The remaining 10 subjects for each cohort are dosed at least 24 hours after the sentinel cohort at a ratio of 9:1 active to placebo.

Sentinel dosing is applied for all dosing cohorts to ensure subject safety as follows:
Two subjects of each cohort are dosed at a ratio of 1:1 (2196+2130:placebo) and then undergo a safety monitoring period for at least 24 hours before dosing the rest of the subjects of that cohort.
Escalation from one dose level to the next dose level is based on review of all available safety data up to and including Day 8. In addition, any available PK data will also be reviewed.

### Cohorts

Approximately 48 healthy adult subjects are randomized to receive either 2196+2130 or placebo across the 4 fixed-dose cohorts as provided below.

Cohort 1a (12 subjects):

300 mg (150 mg of antibody 2196 and 150 mg of antibody 2130) (n=9) or placebo (n=1), administered intramuscularly (IM) (direct deltoid IM)
Sentinel dosing: 300 mg IP (150 mg antibody 2196 and 150 mg antibody 2130) (n=1) or placebo (n=1).
   Cohort 1b (12 subjects):
300 mg (150 mg antibody 2196 and 150 mg antibody 2130) (n=9) or placebo (n=1), administered intravenously (IV)
Sentinel dosing: 300 mg (150 mg antibody 2196 and 150 mg antibody 2130) (n=1) or placebo (n=1).
   Cohort 2 (12 subjects):
1000 mg (500 mg antibody 2196 and 500 mg antibody 2130) (n=9) or placebo (n=2), administered IV
Sentinel dosing: 1000 mg (500 mg antibody 2196 and 500 mg antibody 2130) (n=1) or placebo (n=1).
   Cohort 3 (12 subjects):
3000 mg (1500 mg antibody 2196 and 1500 mg antibody 2130) (n=9) or placebo (n=1), administered IV
Sentinel dosing: 3000 mg (1500 mg antibody 2196 and 1500 mg antibody 2130) (n=1) or placebo (n=1).

### Study Design

After the screening period, each eligible patient is admitted to a Phase I unit on their respective Day −1 (1 day prior to dosing) and discharged on Day 2 (1 day after dosing, after the 24-hour procedures are completed). Subjects are then monitored for approximately one year after dosing for safety, including recording of adverse events (AEs), and serious adverse events (SAEs), and collection of blood samples for anti-drug antibodies (ADAs). Safety and tolerability and immunogenicity endpoints are evaluated.

Neutralizing antibody serum samples are obtained on the following days: Day 8(±1), Day 31(±2), Day 61(±5), Day 91(±5), Day 151(±5), Day 211 (±5), Day 271 (±10), and Day 361 (±10). The neutralizing antibody concentrations of 2196+2130 against SARS-CoV-2 are measured using wild-type SARS-CoV-2 neutralization assays and/or pseudo-virus neutralization assays. 2196+2130 antibody concentrations in nasal fluid are also summarized.

### Results

The placebo and 2196+2130 groups are compared to show that 2196+2130 can results in serum concentrations of 2196+2130 that can functionally inhibition SARS-CoV-2. The groups are also are compared to show administration of 2196+2130 is safe and tolerable when administered intravenously (IV) or intramuscularly (IM). The groups are also compared to show that anti-drug antibody responses are acceptable after administration of 2196+2130.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

1. A method of treating or preventing Coronavirus Disease 2019 (COVID-19) in a subject, the method comprising administering to a subject in need thereof about 300 mg to about 3000 mg of a first antibody or antigen-binding fragment thereof that binds to a spike protein of SARS-CoV-2 and about 300 mg to about 3000 mg of a second antibody or antigen-binding fragment thereof that binds to a spike protein of SARS-CoV-2, wherein
the first antibody or antigen-binding fragment thereof comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and
the second antibody or antigen-binding fragment thereof comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

2. The method of claim 1, comprising administering about 300 mg of the first antibody or antigen-binding fragment thereof and about 300 mg of the second antibody or antigen-binding fragment thereof.

3. The method of claim 1, comprising administering about 500 mg of the first antibody or antigen-binding fragment thereof and about 500 mg of the second antibody or antigen-binding fragment thereof.

4. The method of claim 1, comprising administering about 1500 mg of the first antibody or antigen-binding fragment thereof and about 1500 mg of the second antibody or antigen-binding fragment thereof.

5. A method of treating or preventing Coronavirus Disease 2019 (COVID-19) in a subject, the method comprising administering to a subject in need thereof about 150 mg of a first antibody or antigen-binding fragment thereof that binds to a spike protein of SARS-CoV-2 and about 150 of a second antibody or antigen-binding fragment thereof that binds to a spike protein of SARS-CoV-2, wherein
the first antibody or antigen-binding fragment thereof comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and
the second antibody or antigen-binding fragment thereof comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

6. The method of claim 1, wherein the first antibody or antigen-binding fragment thereof and the second antibody or antigen-binding fragment thereof are administered in separate pharmaceutical compositions.

7. The method of claim 1, wherein the first antibody or antigen-binding fragment thereof and the second antibody or antigen-binding fragment thereof are administered sequentially.

8.-9. (canceled)

10. The method of claim 1 wherein the first antibody or antigen-binding fragment thereof and/or the second antibody or antigen-binding fragment thereof are administered intravenously.

11. The method of claim 1, wherein the first antibody or antigen-binding fragment thereof and/or the second antibody or antigen-binding fragment thereof are administered via intravenous infusion.

12.-13. (canceled)

14. The method of claim 1, wherein the first antibody or antigen-binding fragment thereof and/or the second antibody or antigen-binding fragment thereof are administered intramuscularly.

15.-17. (canceled)

18. The method of claim 1, wherein the subject has an increased risk of COVID 19.

19. The method of claim 1, wherein the subject has been exposed to SARS-CoV-2.

20. The method of claim 1, wherein the subject does not have a known exposure to SARS-CoV-2.

21.-26. (canceled)

27. The method of claim 1, wherein the first antibody or antigen-binding fragment thereof comprises a variable heavy chain (VH) comprising the amino acid sequence of SEQ ID NO:7 and a variable light chain (VL) comprising the amino acid sequence of SEQ ID NO:8; and/or the second antibody or antigen-binding fragment thereof comprises a variable heavy chain (VH) comprising the amino acid sequence of SEQ ID NO:15 and a variable light chain (VL) comprising the amino acid sequence of SEQ ID NO:16.

28.-31. (canceled)

32. The method of claim 1, wherein the first antibody or antigen-binding fragment thereof and/or the second antibody or antigen-binding fragment thereof comprises a heavy chain constant region comprising a YTE mutation and/or a heavy chain constant region comprising a TM mutation.

33.-34. (canceled)

35. The method of claim 1, wherein the first antibody or antigen-binding fragment thereof comprises a heavy chain comprising amino acids 1-460 of SEQ ID NO:24 and a light chain comprising the amino acid sequence of SEQ ID NO:25 and/or wherein the second antibody or antigen-binding fragment thereof comprises a heavy chain comprising amino acids 1-460 of SEQ ID NO:22 and a light chain comprising the amino acid sequence of SEQ ID NO:23.

36. The method of claim 1, wherein the first antibody or antigen-binding fragment thereof and/or the second antibody or antigen-binding fragment thereof is fully human or humanized.

37. (canceled)

38. The method of claim 1, wherein the first antibody or antigen-binding fragment thereof is a full length antibody and/or the second antibody or antigen-binding fragment thereof is a full length antibody.

39. The method of claim 1, wherein the first antibody or antigen-binding fragment thereof is an antigen-binding fragment and/or the second antibody or antigen-binding fragment thereof is an antigen-binding fragment.

40. The method of claim 39, wherein the first and/or second antigen-binding fragment comprises a Fab, Fab′, F(ab′)₂, single chain Fv (scFv), disulfide linked Fv, V-NAR domain, IgNar, IgGΔCH2, minibody, F(ab′)₃, tetrabody, triabody, diabody, single-domain antibody, (scFv)₂, or scFv-Fc.

41.-48. (canceled)

49. A method of preventing or treating Coronavirus Disease 2019 in a subject, the method comprising:
intramuscularly administering to the subject about 300 mg of a first antibody that specifically binds to a spike protein of SARS-CoV-2 and comprises: VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6;
and intramuscularly administering to the subject about 300 mg of a second antibody that specifically binds to the spike protein of SARS-CoV-2 and comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

50.-51. (canceled)

52. The method of claim 49, wherein the first antibody comprises a variable heavy chain (VH) comprising the amino acid sequence of SEQ ID NO:7 and a variable light chain (VL) comprising the amino acid sequence of SEQ ID NO:8; and wherein the second antibody comprises a variable heavy chain (VH) comprising the amino acid sequence of SEQ ID NO:15 and a variable light chain (VL) comprising the amino acid sequence of SEQ ID NO:16.

53. (canceled)

54. The method of claim 49, wherein the first antibody comprises a human IgG1 constant region comprising a YTE mutation and a TM mutation and wherein the second antibody comprises a human IgG1 constant region comprising a YTE mutation and a TM mutation.

55. The method of claim 49, wherein the first antibody comprises a heavy chain comprising amino acids 1-460 of SEQ ID NO:24 and a light chain comprising the amino acid sequence of SEQ ID NO:25 and wherein the second antibody comprises a heavy chain comprising amino acids 1-460 of SEQ ID NO:22 and a light chain comprising the amino acid sequence of SEQ ID NO:23.

56.-58. (canceled)


SEQUENCE LISTING (25 sequences)

SEQ ID NO: 1 - protein, 8 aa, Artificial Sequence
  Clone 2196 Heavy Chain Variable CDR1
GFTFMSSA

SEQ ID NO: 2 - protein, 8 aa, Artificial Sequence
  Clone 2196 Heavy Chain Variable CDR2
IVIGSGNT

SEQ ID NO: 3 - protein, 16 aa, Artificial Sequence
  Clone 2196 Heavy Chain Variable CDR3
AAPYCSSISCNDGFDI

SEQ ID NO: 4 - protein, 7 aa, Artificial Sequence
  Clone 2196 Light Chain Variable CDR1
QSVSSSY

SEQ ID NO: 5 - protein, 3 aa, Artificial Sequence
  Clone 2196 Light Chain Variable CDR2
GAS

SEQ ID NO: 6 - protein, 10 aa, Artificial Sequence
  Clone 2196 Light Chain Variable CDR3
QHYGSSRGWT

SEQ ID NO: 7 - protein, 123 aa, Artificial Sequence
  Clone 2196 Heavy Chain Variable Region
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSS

SEQ ID NO: 8 - protein, 109 aa, Artificial Sequence
  Clone 2196 Light Chain Variable Region
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIK

SEQ ID NO: 9 - protein, 8 aa, Artificial Sequence
  Clone 2130 Heavy Chain Variable CDR1
GFTFRDVW

SEQ ID NO: 10 - protein, 10 aa, Artificial Sequence
  Clone 2130 Heavy Chain Variable CDR2
IKSKIDGGTT

SEQ ID NO: 11 - protein, 22 aa, Artificial Sequence
  Clone 2130 Heavy Chain Variable CDR3
TTAGSYYYDTVGPGLPEGKFDY

SEQ ID NO: 12 - protein, 12 aa, Artificial Sequence
  Clone 2130 Light Chain Variable CDR1
QSVLYSSNNKNY

SEQ ID NO: 13 - protein, 3 aa, Artificial Sequence
  Clone 2130 Light Chain Variable CDR2
WAS

SEQ ID NO: 14 - protein, 8 aa, Artificial Sequence
  Clone 2130 Light Chain Variable CDR3
QQYYSTLT

SEQ ID NO: 15 - protein, 131 aa, Artificial Sequence
  Clone 2130 Heavy Chain Variable Region
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSS

SEQ ID NO: 16 - protein, 112 aa, Artificial Sequence
  Clone 2130 Light Chain Variable Region
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIK

SEQ ID NO: 17-19 - skipped (3 entries)

SEQ ID NO: 20 - protein, 1273 aa, Artificial Sequence
  SARS-CoV-2 Spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 232 aa, Artificial Sequence
  IgG1 sequence comprising triple mutation
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 461 aa, Artificial Sequence
  Clone 2130 Full length heavy chain
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 219 aa, Artificial Sequence
  Clone 2130 Full length light chain
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 453 aa, Artificial Sequence
  Clone 2196 Full length heavy chain
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 216 aa, Artificial Sequence
  Clone 2196 Full lenght light chain
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC